(19) United States
(12) Patent Application Publication
(10) Pub. No.: US 2025/0228471 A1
(43) Pub. Date: Jul. 17, 2025

(54) IMPLANTABLE SENSOR

(71) Applicant: CORNEAT VISION LTD, Ra'anana (IL)

(72) Inventors: Gilad LITVIN, MOSHAV SDE VARBURG (IL); Almog ALEY-RAZ, MOSHAV SDE VARBURG (IL)

(73) Assignee: CORNEAT VISION LTD, Ra'anana (IL)

(21) Appl. No.: 18/841,697

(22) PCT Filed: Feb. 27, 2023
(86) PCT No.: PCT/IL2023/050206

§ 371 Date: Aug. 27, 2024

Related U.S. Application Data
(60) Provisional application No. 63/314,418, filed on Feb. 27, 2022.

(51) Int. Cl. A61B 5/145; A61B 5/00; A61B 5/0205

(52) U.S. Cl. CPC A61B 5/14503; A61B 5/0022; A61B 5/0205

(57) ABSTRACT

The present invention provides an implantable synthetic patch comprising at least one porous polymer and at least one sensor, switch sensor and/or transmitter and uses thereof in the diagnosis of a disease, condition, disorder, or state of a subject in need thereof.

### BACKGROUND OF THE INVENTION

Precision medicine can be defined as the prevention, investigation and treatment of diseases taking individual variability into account. There are multiple ways in which the field of precision medicine may be advanced; however, recent innovations in the fields of electronics and microfabrication techniques have led to an increased interest in the use of implantable biosensors in precision medicine.

Implantable biosensors are an important class of biosensors because of their ability to provide continuous data on the levels of a target analyte; this enables trends and changes in analyte levels over time to be monitored without any need for intervention from either the patient or clinician. As such, implantable biosensors have great potential in the diagnosis, monitoring, management and treatment of a variety of disease conditions.

In most cases, especially when long term monitoring of a subject condition is required with a biosensor that is implantable within the body, the host immune responses elicit unwanted and adverse effect and rejection of the implant, risking the health of the subject and requiring the extraction of the implant and re-implanting another.

There is a continuing need to provide a safe and biocompatible implantable device having the ability to continuously monitor a subject with minimal intervention compared with common procedure in clinical environments.

### SUMMARY OF THE INVENTION

The present invention provides an implantable synthetic patch comprising at least one porous polymer and at least one sensor and/or transmitter.

When referring to a sensor (or transducer) it should be understood to encompass a device that translates at least one physical property (pressure, temperature, humidity, heartbeat, levels of biological compounds found in bodily fluids, such as for example glucose, hormones, insulin and so forth) to an electrical signal. When referring to a transmitter it should be understood to refer to s sensor that conveys data over long distances (for example the data can be read by a distant controller/reading/receiving/receiver device such as a handheld computer, a computer, a phone and so forth). Another type of sensor that can be used in an implantable synthetic patch of the invention is a switch sensor, defined to relate to a sensor that holds a threshold (X) and outputs true or false indications. For example, if measured pressure>(X) output true otherwise output false.

In some embodiments, said at least one sensor and/or transmitter is a wireless sensor and/or transmitter.

In some embodiments, an implantable synthetic patch of the invention further comprises at least one microprocessor. In some embodiments, an implantable synthetic patch of the invention further comprises at least one WiFi and/or Bluetooth transmitter. In some embodiments, an implantable synthetic patch of the invention further comprises at least one receiver.

Thus, the present invention provides an implantable synthetic patch comprising at least one porous polymer and at least one sensor. Furthermore, the present invention provides an implantable synthetic patch comprising at least one porous polymer and at least one transmitter.

The device of the present invention is implantable, and capable of being placed within the human body for a pre-determined time. In some embodiments, device of the invention may be implanted under the skin (transdermal) and/or dermal tissue of a subject (external skin, internal skin tissue, and so forth). In other embodiments, device of the invention may be implanted internally or externally on an organ or parts thereof of a subject (for example-pancreas, heart, uterus, gland, brain, colon, liver, stomach, lung, muscle, and any combinations thereof).

In some embodiments, an implantable synthetic patch of the invention is a biocompatible patch.

In some embodiments, an implantable synthetic patch of the invention is a biodegradable patch.

In some embodiments, an implantable synthetic patch of the invention is a non-biodegradable patch.

In some embodiments, said at least one porous polymer has pores of having pores of less than 5 microns.

In some embodiments, said at least one porous polymer has pores of between 1 to 5 microns.

In some embodiments, said at least one porous polymer has pores of between 5 to 20 microns.

In some embodiments an implantable synthetic patch of the invention has a thickness of between 50 to 250 microns. In other embodiments, the thickness of said patch is between 250 to 2500 microns.

In some embodiments, said at least one sensor and/or transmitter is fully embedded/encapsulated within said polymeric patch comprising at least one porous polymer. In some embodiments, said at least one sensor and/or transmitter is at least partially embedded/encapsulated within said polymeric patch comprising at least one porous polymer. In other embodiments, said implantable synthetic patch of the invention further comprises at least one semi-permeable layer capable of selectively passing at least one biological compound (for example: certain peptides, hormones i.e. insulin, thyroxin, nutrients like glucose, pH, drugs, chemotherapeutic agents) through the pores of said patch.

The term “porous biocompatible layer” should be understood to encompass any type of layer (or film) formed from material that can perform its desired function. This layer should not elicit any undesirable local or systemic effects in the recipient or beneficiary of that therapy while generating the most appropriate beneficial cellular or tissue response in that specific situation and optimizing the clinically relevant performance of that therapy. The biocompatible layer of the device of the invention allows the implanted patch to exist in harmony with tissue it is in contact with without causing deleterious changes. The layer is porous, in some embodiments said layer has pore size of at least at least about 2 μm (when referring to pore size it should be understood to relate to the average pore sizes).

In some embodiments, said porous polymeric structure comprises nanofibers. In other embodiments, said porous polymeric structure comprises at least one porous electrospun polymer. In further embodiments, said porous polymeric structure comprises at least one polymer selected from aromatic polyurethane, polycarbonate, poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof.

In some embodiments, said implantable synthetic patch of the invention further comprising at least one active agent. In some embodiments, said at least one active agent is comprises within said implantable patch of the invention in the form of a metered and/or gated release reservoir.

The term “electrospinning” or “electrospun” or any of its lingual deviations should be understood to encompass a process using an electrical charge to draw very fine (typically on the micro or nano scale) fibers from a liquid. Electrospinning from molten precursors is also practiced; this method ensures that no solvent can be carried over into the final product. The fibers produced using electrospinning processes have increased surface area to volume ratio. Various factors are known to affect electrospun fibers including but are not limited to: solution viscosity, surface tension, electric field intensity and distance.

In a typical electrospinning process a sufficiently high voltage is applied to a liquid droplet of a polymeric material (a polymer solution, a monomeric precursor thereof, sol-gel precursor, particulate suspension or melt), the body of the liquid becomes charged, and electrostatic repulsion counteracts the surface tension and droplet is stretched, at a critical point a stream of liquid erupts from the surface. If the molecular cohesion of the liquid is sufficiently high, stream breakup does not occur (if it does, droplets are electrosprayed) and a charged liquid jet is formed. As the jet dries in flight, the mode of current flow changes from ohmic to convective as the charge migrates to the surface of the fiber. The jet is then elongated by a whipping process caused by electrostatic repulsion initiated at small bends in the fiber, until it is finally deposited on the grounded collector. The elongation and thinning of the fiber that results from this bending instability leads to the formation of uniform fibers with nanometer-scale diameters.

Biocompatible polymers which may be applied in an electrospinning process include but are not limited to poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyvinyl chloride (PVC), hyaluronic acid (HA), chitosan, alginate, polyhydroxybuyrate and its copolymers, Nylon 11, Cellulose acetate, hydroxyappetite, or any combination thereof. Polymers include but are not limited to poly(3-hydroxybutyric acid-co-3-hydroxyvaleric acid), poly(DL-lactide), poly urethane, polycaprolactone, and poly(L-lactide) or any combination thereof.

Electrospun fibers are typically several orders in magnitude smaller than those produced using conventional spinning techniques. By optimizing parameters such as: i) the intrinsic properties of the solution including the polarity and surface tension of the solvent, the molecular weight and conformation of the polymer chain, and the viscosity, elasticity, and electrical conductivity of the solution; and ii) the operational conditions such as the strength of electric field, the distance between spinneret and collector, and the feeding rate of the solution, electrospinning is capable of generating fibers as thin as tens of nanometers in diameter. Additional parameters that affect the properties of electrospun fiber include the molecular weight, molecular-weight distribution and structure (branched, linear etc.) of the polymer, solution properties (viscosity, conductivity and surface tension), electric potential, flow rate and concentration, distance between the capillary and collection screen, ambient parameters (temperature, humidity and air velocity in the chamber), motion of target screen (collector) and so forth. Fabrication of highly porous fibers may be achieved by electrospinning the jet directly into a cryogenic liquid. Well-defined pores developed on the surface of each fiber as a result of temperature-induced phase separation between the polymer and the solvent and the evaporation of solvent under a freeze-drying condition.

Several approaches have been developed to organize electrospun fibers into aligned arrays. For example, electrospun fibers can be aligned into a uniaxial array by replacing the single-piece collector with a pair of conductive substrates separated by a void gap. In this case, the nanofibers tend to be stretched across the gap oriented perpendicular to the edges of the electrodes. It was also shown that the paired electrodes could be patterned on an insulating substrate such as quartz or polystyrene so the uniaxially aligned fibers could be stacked layer-by-layer into a 3D lattice. By controlling the electrode pattern and/or the sequence for applying high voltage, it is also possible to generate more complex architectures consisting of well-aligned nanofibers.

Electrospun nanofibers could also be directly deposited on various objects to obtain nanofiber-based constructs with well-defined and controllable shapes. In addition, one can manually process membranes of aligned or randomly oriented nanofibers into various types of constructs after electrospinning: for example, fabrication of a tube by rolling up a fibrous membrane or the preparation of discs with controllable diameters by punching a fibrous membrane.

The present invention relates to any eletrospinning technique known in the art, which includes Electrospinning, J. Stanger, N. Tucker, and M. Staiger, I-Smithers Rapra publishing (UK), An Introduction to Electrospinning and Nanofibers, S. Ramakrishna, K. Fujihara, W-E Teo, World Scientific Publishing Co. Pte Ltd (June 2005), Electrospinning of micro- and nanofibers: fundamentals and applications in separation and filtration processes, Y. Fillatov, A. Budyka, and V. Kirichenko (Trans. D. Letterman), Begell House Inc., New York, USA, 2007, which are all incorporated herein by reference in their entirety.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference. It is to be understood that although the according to the presently preferred embodiment of the invention is described with a particular emphasis to the electrospinning technique, it is not intended to limit the scope of the invention to the electrospinning technique. Representative examples of other spinning techniques suitable for the present embodiments include, without limitation, a wet spinning technique, a dry spinning technique, a gel spinning technique, a dispersion spinning technique, a reaction spinning technique or a tack spinning technique. Such and other spinning techniques are known in the art and disclosed, e.g., in U.S. Pat. Nos. 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

In some embodiments, said at least one active agent is selected from a protein, collagen, fibronectin, or TGF-beta 2, heparin, growth factors, antibodies, antimetabolites, chemotherapeutic agents, anti-inflammatory agent, antibiotic agent, antimicrobial agent and any combinations thereof.

In some embodiments, said at least one sensor is a biosensor. In some embodiments, said at least one sensor is a bio-transmitter (i.e. a sensor and/or transmitter that is adapted to sense at least one biological property).

Said biosensor being an analytical device, used for the detection of a chemical substance, that combines a biological component with a physicochemical detector. The sensitive biological element, e.g. tissue, microorganisms, organelles, cell receptors, enzymes, antibodies, nucleic acids, etc., is a biologically derived material or biomimetic component that interacts with, binds with, or recognizes the analyte under study. The biologically sensitive elements can also be created by biological engineering. The transducer or the detector element, which transforms one signal into another one, works in a physicochemical way: optical, piezoelectric, electrochemical, electrochemiluminescence etc., resulting from the interaction of the analyte with the biological element, to easily measure and quantify. In some embodiments, a biosensor reader device connects with the associated electronics or signal processors that are primarily responsible for the display of the results.

A biosensor typically consists of a bio-receptor (enzyme/antibody/cell/nucleic acid/aptamer), transducer component (semi-conducting material/nanomaterial), and electronic system which includes a signal amplifier, processor and display. Transducers and electronics can be combined, e.g., in CMOS-based microsensor systems. The recognition component, often called a bioreceptor, uses biomolecules from organisms or receptors modeled after biological systems to interact with the analyte of interest. This interaction is measured by the bio-transducer which outputs a measurable signal proportional to the presence of the target analyte in the sample. The general aim of the design of a biosensor is to enable quick, convenient testing at the point of concern or care where the sample was procured.

In some embodiments, said at least one sensor is selected from a sensor for Oxygen levels (local and systemic), glucose levels, Glomerular Filtration Rate (GFR), Blood Pressure (BP), heart rhythm, cancer marker levels: alpha feto protein, CA125, CA15-3, CA 19-9, CEA, PSA, hCG or beta hCG, hormone levels: insulin, glucagon, adrenalin, somatostatin, corticosteroids, thyroid hormones, Leptin, Adiponectin, Histamine, sex hormones: Testosterone, Estrogen, Progesterone, Androstenedione, hypothalamus-hypophysis agents: TSH, ACTH, ADH (Vasopressin), LH, FSH, GH, TRH (Thyrotropin Releasing Hormone), GnRH (Gonadotropin Releasing Hormone), GHRH (Growth Hormone Releasing Hormone), CRH (Corticotropin releasing hormone) and neurotransmitter levels: Dopamine, Serotonin, Noradrenaline, Acetyl Choline, Gama Amino Butyric Acid (GABA), Galanin, Encephalin, Neuro peptide Y, Opioids, Endorphins, Aspartate, D-serine, Glutamate, Glycine, Nitric Oxide, Oxytocin, and drug level: Lithium, Tricyclic antidepressants, Antibiotics, Antipsychotics, Anti-epileptics, Anti-Cancer and any combinations thereof.

The invention further provides an implantable synthetic patch according to any one of the preceding claims, for use in the diagnosis of at least one condition, disease, disorder or state of a subject in need thereof.

The invention further provides a method of diagnosing at least one condition, disease, disorder or state of a subject in need thereof, said method comprising the step of providing an implantable synthetic patch according to any one of the preceding claims and implanting said implantable synthetic patch in said subject.

The invention further provides an implantable synthetic patch according to any one of the preceding claims, for use in the treatment of at least one condition, disease, disorder or state of a subject in need thereof; wherein said implantable synthetic patch comprises at least one active agent (as disclosed herein above and below).

The invention further provides a method of treating at least one condition, disease, disorder or state of a subject in need thereof, said method comprising the step of providing an implantable synthetic patch according to any one of the preceding claims, wherein said implantable synthetic patch comprises at least one active agent (as disclosed herein above and below), and implanting said implantable synthetic patch in said subject.

In some embodiments said at least one condition, disease, disorder or state is selected from diabetes, high blood pressure, arrythmia, cancer, depression, drug addiction, hormone deficiency, menopause, mineral deficiency, osteoporosis, brain functions, neurological disorders, inflammation, toxin levels, and so forth and any combinations thereof.

In some embodiments, said implantable synthetic patch of the invention comprises at least one receiver capable of receiving a signal to enable the release of at least one active agent from a patch of the invention. In some embodiments, said release of said at least one active agent is controlled release. In other embodiments, said release of said at least one active agent is the result of a detection of at least one biological property by said sensor, the receiving of said transmittal of signal by an external receiving device (external of said patch of the invention) and receiving a signal for release of said at least one active agent by said at least one receiver of said patch of the invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows an implantable sensor of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

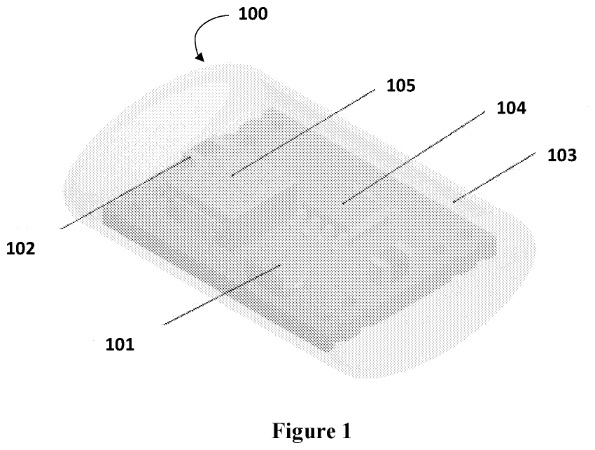

FIG. 1 shows an implantable sensor of the present invention (100), having an encapsulating/embedding porous polymer patch (103), a sensor (101), a WiFi and/or Bluetooth transmitter (102), a microprocessor (104), a compartment comprising an active agent (105).

### Example 1—Coated RFID Sensor of the Invention-Subcutaneous Implantation in Rats

### Safety and Performance Assessment of a Coated RFID Sensor in a Rat Model

Study Objective: Evaluate the safety and performance of an RFID sensor coated with electrospun material following subcutaneous implantation in SD rats

Operation: Each animal are subjected to a subcutaneous implantation in the dorsal flank area where one side are implanted with the Test Device and the other with the Control Device (a non-coated RFID sensor). Implantation sites are marked with a tattoo or a permanent marker.

Examinations: Individual detailed clinical signs including local reaction at both implantation sites: Once weekly. Body Weights: Once weekly. Cage-side observations: Once daily including weekends. RFID readings of both implants-once weekly.

Study Duration: 4 weeks

Post-operative Care: Analgesia is administered for up to 2 days post-surgery (per designated veterinarian discretion). Antibiotics prophylaxis treatment is administered up to 3 days post-op (per designated veterinarian discretion).

Terminal Investigations: Animals are euthanized, and implantation sites are harvested, and any implant's migration are recorded. The implanted devices are gently removed and immediately fixed in formalin solution (care is taken to avoid inflicting damage to the tissue-implant interface). Histopathological processing of all implantation sites: medial sectioning, Paraffin embedding, H&E staining (total of 12 slides).

Histopathological evaluation for all slides should include, but not limited to, the following: Extent of fibrosis/fibrous capsule and inflammation; Degeneration as determined by changes in tissue morphology; Severity of inflammatory response and cell types; The presence and extent of necrosis; Other tissue alterations such as vascularization, fatty infiltration and granuloma formation.

1. An implantable synthetic patch comprising at least one porous polymer and at least one sensor and/or transmitter.

2. An implantable synthetic patch of claim 1, having a porous polymeric structure having pores of less than 5 microns.

3. An implantable synthetic patch of claim 1, having a porous polymeric structure with pores of between 5 to 20 microns.

4. An implantable synthetic patch according to claim 1, being a biocompatible patch.

5. An implantable synthetic patch according to claim 1, being a biodegradable patch.

6. An implantable synthetic patch according to claim 1, being a non-biodegradable patch.

7. An implantable synthetic patch according to claim 1, having a thickness of between 50 to 250 microns.

8. An implantable synthetic patch according to claim 1, having a thickness of between 250 to 2500 microns.

9. An implantable synthetic patch according to claim 1, wherein said porous polymeric structure comprises nanofibers.

10. An implantable synthetic patch according to claim 1, wherein said porous polymeric structure comprises at least one porous electrospun polymer.

11. An implantable synthetic patch according to claim 1, wherein said porous polymeric structure comprises at least one polymer selected from aromatic polyurethane, polycarbonate, poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof.

12. An implantable synthetic patch according to claim 1, further comprising at least one active agent.

13. An implantable synthetic patch according to claim 1 further comprising at least one active agent selected from a protein, collagen, fibronectin, or TGF-beta 2, heparin, growth factors, antibodies, antimetabolites, chemotherapeutic agents, anti-inflammatory agent, antibiotic agent, antimicrobial agent and any combinations thereof.

14. An implantable synthetic patch according to claim 1, being a transdermal patch.

15. An implantable synthetic patch according to claim 1, wherein said at least one sensor is a biosensor.

16. An implantable synthetic patch according to claim 1, wherein said at least one sensor is selected from a sensor for oxygen levels (local and systemic), glucose levels, Glomerular Filtration Rate (GFR), blood pressure (BP), heart rhythm, cancer marker levels: alpha feto protein, CA125, CA15-3, CA 19-9, CEA, PSA, hCG or beta hCG, hormone levels: insulin, glucagon, adrenalin, somatostatin, corticosteroids, thyroid hormones, Leptin, Adiponectin, Histamine, sex hormones: Testosterone, Estrogen, Progesterone, Androstenedione, hypothalamus-hypophysis agents: TSH, ACTH, ADH (Vasopressin), LH, FSH, GH, TRH (Thyrotropin Releasing Hormone), GnRH (Gonadotropin Releasing Hormone), GHRH (Growth Hormone Releasing Hormone), CRH (Corticotropin releasing hormone) and neurotransmitter levels: Dopamine, Serotonin, Noradrenaline, Acetyl Choline, Gama Amino Butyric Acid (GABA), Galanin, Encephalin, Neuro peptide Y, Opioids, Endorphins, Aspartate, D-serine, Glutamate, Glycine, Nitric Oxide, Oxytocin, and drug level: Lithium, Tricyclic antidepressants, Antibiotics, Antipsychotics, Anti-epileptics, Anti-Cancer and any combinations thereof.

17. A method of diagnosis of at least one condition, disease, disorder or state of a subject in need thereof, said method comprising implanting an implantable synthetic patch comprising at least one porous polymer and at least one sensor and/or transmitter.
